(19) United States
(12) Patent Application Publication
(10) Pub. No.: US 2011/0152343 A1
(43) Pub. Date: Jun. 23, 2011

(54) PROTEASE INHIBITORS AND BROAD-SPECTRUM ANTIVIRAL

(75) Inventors: M. JAVAD AMAN, Gaithersburg, MD (US); Michael Kinch, Laytonsville, MD (US); Sven Enterlein, Gaithersburg, MD (US); Yunus Abdul, Gaithersburg, MD (US)

(73) Assignee: Functional Genetics, Inc., Gaithersburg, MD (US)

(21) Appl. No.: 12/644,402

(22) Filed: Dec. 22, 2009

(51) Int. Cl. A61K 31/381; C07D 407/06; C07D 333/24; A61K 31/343; C12Q 1/70

(52) U.S. Cl. 514/438; 549/467; 549/74; 514/469; 435/5

(57) ABSTRACT

Caspace inhibition provides inhibition of viral infection across a wide collection of caspaces and viruses. Caspace inhibition, the prevention of the formation of active caspaces, can be achieved either through gene therapy, protein binding an inhibition, or through small molecule administration. Examples for small molecule inhibition allow the formation of a pharmacaphore to identify more and more active small molecules.

### BACKGROUND

1. Field of the Invention

The present invention relates to a set of compositions comprising caspase inhibitors that are further effective at preventing, treating, and/or managing viral infection caused by a variety of virus types. The present invention further relates to methods of administration of compositions of the present invention to a patient or animal experiencing or at risk of viral infection as well as methods for screening additional compounds to identify caspase inhibitors that are further effective at inhibiting viral infection.

2. Related Art

Viruses have long been known to be the causative agent in a wide variety of human and animal infectious diseases associated with human and animal morbidity and mortality. Many different viral pathogens have consistently caused debilitating or fatal diseases in humans and animals (e.g., influenza, etc.) while others are emerging or re-emerging (e.g., HIV, West Nile virus, SARS, etc.).

Efforts to treat or prevent viral infection in both animals and humans may be generally classified into two broad categories: vaccines and antiviral drugs. Vaccines generally work by priming the immune system of an individual through administration of an immunogen. The immunogen is typically either a killed virus, an attenuated virus, or a viral subunit that is incapable of causing infection but is sufficient to trigger an immune response. Since the immunogen resembles the live virus targeted by the vaccination, the immune system is able to readily identify and eliminate the virus during early stages of actual infection. When available, vaccines are very effective at immunizing individuals against particular viruses that cause disease.

However, vaccines are often limited in that they are generally only effective in immunizing individuals prior to infection (i.e., they are ineffective as a means for treating infected individuals that may or may not yet be experiencing symptoms of disease). Furthermore, vaccines are often ineffective in vaccinating individuals against viruses that are highly mutable since these viruses are able to evade any immunity generated by vaccination.

Research has also focused on developing antiviral medications as a means for treating individuals who are already infected as well as treating or preventing viral disease where vaccination methods are seen as unavailable or unlikely. Such approaches toward developing antiviral medications have generally sought to identify molecules or drugs that interfere with the basic mechanisms or steps of viral infection, through what is called “rational drug design.” Alternatively, antiviral drugs, such as interferons or antibodies, may instead be designed to broadly stimulate the immune system against a range of pathogens.

In general, viruses proceed through a series of steps akin to the following during their normal infection cycle: (1) attachment (i.e., specific binding between viral capsid or coat proteins and receptors on the host cell surface), (2) penetration (i.e., entry into the host cell generally through endocytosis or membrane fusion), (3) uncoating (i.e., digesting or degrading the viral coat to allow the contents and viral genome to be released into the cell), (4) replication and assembly (i.e., the synthesis of new viral proteins and DNA/RNA, including intermediates, necessary to form new virus particles), (5) maturation (i.e., post-translational modification and processing to form mature virus particles), and (6) release or budding (i.e., freeing the virus particles to infect new host cells). However, not all viruses proceed through all of these steps in the manner summarized. For example, HIV undergoes maturation after being released from the host cell.

Despite noted success in the design and development of novel antiviral drugs in recent years, including the development of protease inhibitors, existing therapies are limited in terms of the number and breadth of viruses that they may be used to treat. In addition, many strains of viruses have become resistant to antiviral drugs as a result of mutation of their viral genomes. Accordingly, there continues to be a need in the art for the development of new classes of antiviral drugs to treat or prevent viral disease, especially those that show promise against a variety of virus types. There also continues to be a need in the art for the development of novel antiviral drugs that are effective against highly mutable viruses that are generally capable of evading treatment via existing vaccines and drugs.

### BRIEF DESCRIPTION OF THE DRAWINGS

The invention will be described in conjunction with the accompanying drawings, in which:

FIG. 1 shows the effects of various FGI-103 compounds on the activity of different caspase enzymes.

FIG. 2 shows the effectiveness of using a representative FGI-103 compound, NSC 369723, to counteract infection by RSV depending on the timing of administration.

### DETAILED DESCRIPTION OF THE PREFERRED EMBODIMENTS

### Definitions

It is advantageous to define several terms before describing the invention. It should be appreciated that the following definitions are used throughout this application.

For the purposes of the present invention, the term “pharmacophore” refers to a molecular framework that carries the essential features responsible for a biological activity of a drug or compound. Stated differently, the term “pharmacophore” may also refer to an ensemble of steric and electronic features that is necessary to ensure interaction with a specific biological target to trigger (or block) its biological response. In some cases, the term “pharmacophore” may refer to a portion of a compound known to have a desired biological activity with nonessential or unnecessary atoms and/or constituents removed.

For the purposes of the present invention, the term “mammal” is intended to include, for example, any human, monkey, or other primate. The term “mammal” further refers to other animals having an agricultural purpose or domesticated use including, but not limited to, cattle, sheep, goats, pigs, horses, canines, cats, etc. The term “mammal” also refers to animals having research or laboratory uses including, but not limited to, rabbits, mice, rats, etc. The term “animal” more broadly refers to any animal, including vertebrates and mammals, having commercial, agricultural, domesticated, research, and/or industrial usefulness.

For the purposes of the present invention, the term “inhibitor” refers to any molecule or compound, or any combination thereof, which inhibits the enzymatic activity of one or more caspase enzymes present within a host cell. The term “inhibitor” may also refer to any such caspase inhibitors that further interfere with viral infection of a host cell by a virus Inhibitors may include small molecules which bind to a caspase enzyme and inhibit its activity Inhibitors may also include polynucleotides that may be used for antisense or RNAi approaches, such as antisense RNA, dsRNA, siRNA, etc., as a means to down-regulate the level and/or activity of one or more caspase enzymes in a host cell. It is envisioned that the term “inhibitor” may further encompass any reagent used to deliver such antisense or RNAi-mediated polynucleotides to a host cell, such as DNA transfection vectors, homologous recombination vectors, and/or viral vectors to stably or transiently transfect a host cell or to stably incorporate into the genome of a host cell, such as by gene therapy methods.

For the purposes of the present invention, the term “host cell” refers to any animal or human cell that is experiencing, subject to, or at risk of viral infection caused by a virus. A “host cell” may be cultured in vitro or exist in situ within the tissue of a living person or animal that is experiencing, subject to, or at risk of viral infection caused by a virus.

### DESCRIPTION

During infection, viruses hijack the cellular machinery to effectively replicate the viral genome and produce new virus particles. Historically, most efforts to develop therapeutic agents against viral infection have been directed against the virus itself, rather than components of the host cell that are utilized during infection. While this approach has been successful in developing many life-saving antiviral drugs, the usefulness of direct antiviral targeting is limited by the extreme plasticity of some viral genomes allowing viruses to evade the effects of antiviral compounds under selection pressure. Furthermore, recent advances in molecular biology may allow for the engineering or selection of unnatural viruses (i.e., bioweapons) that are designed to evade conventional therapies.

An alternative approach is to develop compounds or therapies that target host cell components to deny the ability of viruses to cause disease or damage to a host. This approach has a number of advantages. First, many different types of viruses utilize a relatively small number of host cell components and mechanisms providing opportunities for broad-spectrum targeting of viruses. Second, by targeting host cell components, it becomes more difficult if not impossible for highly mutable viruses (e.g., influenza and HIV) to evade and become resistant to such compounds leading to more durable therapies. Third, many virally-hijacked host pathways are highly conserved among different human and animal host species allowing such antiviral compounds to be effective in treating, preventing, and/or managing viruses in a variety of different hosts. A well-known example of this latter concept is evidence that influenza virus can shuttle among humans, pigs and avian species.

Most of the current emphasis on host targeting emphasizes modulation of either the host immune response or cellular receptors for viral binding to the host cells. However, much less effort has been made to develop compounds or therapies that target intracellular host cell components or other cellular components not involved per se in recognition, binding and/or attachment of viruses to host cells.

Work at Functional Genetics, Inc. (FGI) has identified a family of small molecules based around a common pharmacophore that are collectively part of the FGI-103 program. Further work has demonstrated that these molecules have the ability to inhibit the propagation of multiple and different types of viruses including, but not limited to, DNA viruses, positive and negative strain viruses, and retroviruses. For example, it has been shown that these identified FGI-103 molecules inhibit infection by RSV, PIV, Pox, Ebola, Marburg, Rift Valley Fever, Lassa Fever, PRRS, and other viruses. In addition, these molecules have also shown potential antiviral activity in both contexts of preventing or treating viral infection.

Such broad-spectrum antiviral activity exhibited by these FGI-103 compounds led researchers at FGI to postulate that the molecules were inhibitors that targeted a component of a host pathway commonly shared or utilized by a variety of virus types during their normal infection cycle rather than a viral target. To determine potential host-cell targets of the identified FGI-103 inhibitors, researchers at FGI conducted a three dimensional query based on the known pharmacophore substructure shared by the FGI-103 compounds. This query identified similarities with benzamidine and APMSF, both of which are known to possess protease inhibitory activity.

Based on these findings, a variety of protease family enzymes were tested for the ability of the identified FGI-103 molecules to decrease their enzymatic activity. Surprisingly, these tests showed that the FGI-103 molecules inhibited protease activity of caspase family members. Although none of the FGI-103 compounds functioned as a pan-caspase inhibitor, each of the FGI-103 compounds blocked activity of different caspases to varying extents. Taken together, however, the FGI-103 compounds demonstrated an ability to inhibit the activity of nine different caspases. Furthermore, as described in greater detail below, FGI-103 compounds appear to block early events in viral infection of host cells suggesting that compounds described herein may be useful in both preventing as well as treating or managing viral infection.

Proteases may be generally classified into six groups: serine proteases, cysteine proteases, threonine proteases, aspartic acid proteases, metalloproteases, and glutamic acid proteases. Caspases comprise a family of cysteine proteases having a nucleophilic cysteine residue in the active site of the enzyme. At present, at least 14 mammalian caspases have been identified; however, additional caspases continue to be discovered. See, e.g., Fan, T., et al., Caspase Family Proteases and Apoptosis. Acta Biochimica et Biophysica Sinica 37(11): 719-27 (2005), the disclosure of which is hereby incorporated by reference. In general, caspase-2, -8, -9, and -10 are understood as activators or initiators of apoptosis, whereas caspase-3, -6, and -7 are understood as effectors or executioners of apoptosis. Many of the other remaining caspases are believed to function as mediators of inflammation and cytokine maturation. However, these functions may not be fully distinct or exclusive of one another, and each caspase member may be capable of significant overlap in function.

Generally speaking, caspases are normally present in cells as inactive precursor enzymes (zymogens) having little or no protease activity. Caspases tend to have a similar domain structure comprising a pro-peptide followed by a large and a small subunit. The pro-peptide is usually either a caspase recruitment domain (CARD) or a death effector domain (DED). Upon triggering initiating events, caspases become activated at least in part by proteolytic processing of the caspase between the large and small subunits to form a heterodimer. This processing step of the caspase zymogen causes rearrangement into an active conformation. Activated caspases typically function as heterotetramers formed by dimerization of two caspase heterodimers. See, e.g., Taylor, et al., Apoptosis: controlled demolition at the cellular level. Nat Rev Mol Cell Biol. 9: 231-41 (2008), the disclosure of which is hereby incorporated by reference.

Caspases cleave target protein molecules based on a set of preferred substrate tetrapeptide sequences. See, e.g., Wee, et al., SVM-based prediction of caspase substrate cleavage sites, BMC Bioinformatics, 7 (Supp 5): S 14 (2006), the disclosure of which is hereby incorporated by reference. In general, it has been shown that caspases preferentially cleave substrate protein targets at X₁EX₂D tetrapeptide sequences where X₁ and X₂ are limited. (Indeed, the name “caspase” is derived from “cysteinyl aspartic acid-specific proteases” in reference to the fact that caspases are cysteine proteases that cut substrates on the carboxy-terminal side of Asp (D) residues.) Consistent with the X₁EX₂D consensus sequence, caspases have been further categorized into three classes according to their specific tetrapeptide sequence preferences: Group I caspases (e.g., caspase-1, -4, and -5) recognize a (W/L)EHD sequence; Group II caspases (i.e., caspase-2, -3, and -7) recognize a DEXD sequence; and Group III caspases (e.g., caspase-6, -8, -9, and -10) recognize a (L/V)E(T/H)D sequence. However, it is to be understood that these tetrapeptide sequences only represent known preferential sequences. It remains possible that caspases, including those listed above, may further recognize and/or cleave additional sequences, including sequences that are perhaps dissimilar from those described above. In addition, caspases of one group may further cleave substrates having the preferred target sequence of a different group.

Caspases have traditionally been studied in connection with their role as effectors of apoptosis and inflammation. However, recent evidence has also pointed to additional roles for caspases in other cellular processes, such as cellular proliferation and cell-cycle progression. See, e.g., Los M., et al., Caspases: more than just killers? Trends Immunol. 22(1): 31-4 (2001); Algeciras-Schimnich A, et al., Apoptosis-independent functions of killer caspases. Curr Opin Cell Biol. 14(6): 721-6 (2002); Launay S., et al., Vital functions for lethal caspases. Oncogene 24(33): 5137-48 (2005), the disclosures of which are hereby incorporated by reference. Current research at FGI, described herein as a basis in part for the present invention, suggests yet another role for caspases, wherein the protease activity of caspases is utilized by viruses during their infection cycle. Indeed, caspase activity appears to be critical or essential for viral infection by a variety of virus types in light of evidence, described herein, showing that FGI-103 caspase inhibitors interfere with and/or block the ability of viruses to successfully infect host cells. Therefore, it is generally proposed herein that various compositions and methods for inhibiting caspases may be effective in preventing, treating, and/or managing viral infection and disease.

According to one broad aspect of the present invention, compositions are provided comprising a caspase inhibitor, or combination thereof, for the treatment, prevention, and/or management of infection by a virus or combination of viruses in a mammal According to some embodiments, caspase inhibitors of the present invention may include any of the small molecules disclosed in U.S. patent application Ser. Nos. 11/464,001 and 11/464,007, or a combination thereof, the disclosures of which are hereby incorporated by reference in their entirety. According to another set of embodiments, caspase inhibitors of the present invention may include any small molecules within the FGI-103 family as disclosed in related U.S. patent application Ser. Nos. 11/952,421, 60/982,227, and 60/884,928, the disclosures of which are hereby incorporated by reference in their entirety. More particularly, such FGI-103 small molecule compounds of the present invention may include NSC compounds 294199, 300510, or 369723 as well as BG11 or BG17, or a combination thereof.

According to a broader set of embodiments, compositions of the present invention may include any small molecule having an identical or similar pharmacophore common to the FGI-103 family of molecules or compounds identified herein or incorporated by reference in the present application. Such molecules may further have an identical or similar pharmacophore as the current lead FGI-103 drug candidate, NSC 369723. According to another broad set of embodiments, compositions of the present invention may comprise any small molecule that is shown to inhibit capase activity assuming such molecule is further shown to inhibit viral infection of a host cell and/or block viral disease.

Compositions of the present invention may further comprise a pharmaceutical composition comprising a therapeutically effective amount of any of the small molecules (or combinations of small molecules) described above together with other materials, such as a suitable carrier, excipient, etc., for administration to a human or animal experiencing a viral infection or at risk of a viral infection. Such pharmaceutical compositions may be either in solid or liquid form and may be administered as appropriate to an individual parenterally, topically, orally, or through mucosal surfaces and routes. The exact dosage corresponding to a therapeutically effective amount will vary from mammal to mammal and virus to virus. As a general range for humans, 0.01 mg/kilo/day-50 mg/kilo/day are target dosages. Those of skill in the art are well equipped by conventional protocols, given the identification of targets and compounds herein, to identify specific dosages for specific mammals, specific viruses, and specific modes of administration. See, e.g., “Remington: The Science and Practice of Pharmacy,” University of the Sciences in Philadelphia, 21st ed., Mack Publishing Co., (2005), the disclosure of which is hereby incorporated by reference in its entirety.

A “therapeutically effective” amount of the inventive compositions can be determined by prevention or amelioration of viral infection of host cells or viral disease in a patient or animal. It will be understood that, when administered to a human patient, the total daily usage of the agents or composition of the present invention will be decided by the attending physician within the scope of sound medical judgment. The specific therapeutically effective dose level for any particular patient will depend upon a variety of factors: the type and degree of the cellular or physiological response to be achieved; activity of the specific composition employed; the specific agents or composition employed; the age, body weight, general health, sex and diet of the patient; the time of administration, route of administration, and rate of excretion of the composition; the duration of the treatment; drugs used in combination or coincidental with the composition; and like factors well known in the medical and veterinary arts. For example, it is well within the skill of the art to start doses of the agents at levels lower than those required to achieve the desired therapeutic effect and to gradually increase the dosages until the desired effect is achieved.

According to another broad aspect of the invention, compositions are provided comprising an antisense, siRNA, and/or dsRNA polynucleotide(s) that inhibit caspase function through down-regulation of target caspases to disrupt the ability of viruses to infect host cells and reproduce themselves. For example, compositions of the present invention may comprise an antisense molecule(s) comprising a polynucleotide sequence complementary to a sequence identical or homologous to all or a portion of a caspase gene or coding sequence. Such antisense polynucleotides need not be 100% complementary to a caspase target sequence to hybridize with such target. For example, according to some embodiments, antisense polynucleotide sequences for compositions of the present invention may be anywhere between 70% and 100% complementary to a caspase target sequence, such as at least 70%, 80%, or 90% complementary. One skilled in the art can readily predict substitutions in antisense sequences which are likely to maintain such complementarity and hybridization with target mRNA.

RNA interference (“RNAi”) refers to the process of sequence-specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The process of RNAi begins by the presence of a long dsRNA in a cell, wherein the dsRNA comprises a sense RNA having a sequence homologous to the target gene mRNA and antisense RNA having a sequence complementary to a homologous sequence of the sense RNA. The presence of dsRNA stimulates the activity of a ribonuclease III enzyme referred to as Dicer. Dicer is involved in the processing of the dsRNA into short pieces of dsRNA known as short interfering RNAs (siRNAs) (Berstein et al., 2001, Nature, 409, 363). Short interfering RNAs derived from Dicer activity are typically about 21 to about 23 nucleotides in length and comprise about 19 base pair duplexes (Elbashir et al., 2001, Genes Dev., 15, 188). siRNAs in turn stimulate RNA-induced silencing complex (RISC) by incorporating one strand of siRNA into the RISC and directing degradation of the homologous mRNA target.

Accordingly, compositions of the present invention may comprise dsRNA and/or siRNA polynucleotide molecules capable of eliciting RNAi-mediated downregulation of caspase targets to inhibit viral infectivity of host cells. Such caspase gene or coding sequence may be derived from any caspase gene believed to be involved in viral infection. Such dsRNA and/or siRNA molecules may comprise all or a portion of a sequence identical or homologous to a human or animal caspase gene or coding sequence known in the art (sense strand) in combination with a sequence complementary thereto (antisense strand). The degree of complementarity between the sense and antisense strands of dsRNA or siRNA may be less than 100%, such as at least 80% or 90% complementary, as long as sufficient complementarity exists to maintain sufficient hybridization to elicit RNAi events. According to some preferred embodiments, the dsRNA is expressed as a single polynucleotide molecule having two self-complementary sequences connected by a hairpin loop or other spacer sequence. Such self-complementary polynucleotides may then become processed by host cells into siRNA to trigger RNAi-mediated downregulation of the target caspase.

Preferably, each of said siRNA and/or dsRNA polynucleotide strands is 30 nucleotides or less in length because longer dsRNAs (i.e., dsRNA greater than 30 nucleotides) tend to elicit interferon responses, resulting in nonspecific mRNA degradation and inhibition of protein synthesis. See, e.g., Elbashir et al., Nature 411, 494-498 (2001). According to preferred embodiments, both strands are from about 19 nucleotides to about 29 nucleotides. According to some embodiments, however, dsRNAs and/or siRNAs of the present invention may further include additional non-complementary sequences.

The antisense, dsRNA, and/or siRNA compositions of the present invention may be delivered either directly to an individual or in combination with a delivery reagent. Suitable delivery reagents for administration may include, for example, Mirus Transit TKO lipophilic reagent, lipofectin, lipofectamine, cellfectin, polycations (e.g., polylysine), liposomes, or other similar delivery reagents. Delivery reagents for antisense, dsRNA, and/or siRNA compositions of the present invention may further comprise one or moieties that are linked or associated with such compositions to aid their delivery to particular cells or tissues, such as cells or tissues that are virally infected or at risk of viral infection.

A preferred delivery reagent for the present antisense, dsRNA, and siRNA compositions is through the use of liposomes. Liposomes include emulsions, foams, micelles, insoluble monolayers, liquid crystals, phospholipid dispersions, lamellar layers and the like. In these preparations the composition of the invention to be delivered is incorporated as part of a liposome, alone or in conjunction with a molecule which binds to a desired target, such as antibody, or with other therapeutic or immunogenic compositions. Thus, liposomes either filled or decorated with a desired composition of the invention of the invention can delivered systemically, or can be directed to a tissue of interest, where the liposomes then deliver the selected therapeutic/immunogenic polypeptide compositions.

Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. Ann. Rev. Biophys. Bioeng. 9:467 (1980), U.S. Pat. Nos. 4,235,871, 4,501,728, 4,837,028, and 5,019,369, incorporated herein by reference.

A liposome suspension containing a composition of the invention may be administered intravenously, locally, topically, etc. in a dose which varies according to, inter alia, the manner of administration, the composition of the invention being delivered, and the stage of the disease being treated. The liposomes encapsulating the present dsRNAs and/or siRNAs can be modified so as to avoid clearance by the mononuclear macrophage and reticuloendothelial systems, for example by having opsonization-inhibition moieties bound to the surface.

A variety of different techniques may be employed to produce antisense, dsRNA, or siRNA molecules either by chemical synthesis or by recombinant techniques. For example, recombinant vectors or plasmids may be introduced to cells to achieve expression of such molecules or strands. Antisense, dsRNA, or siRNA molecules may be further synthesized and/or modified by methods well established in the art, such as those described in “Current protocols in nucleic acid chemistry”. Beaucage, S. L. et. al. (Edrs.), John Wiley & Sons, Inc., New York, N.Y., USA, which is hereby incorporated herein by reference, to enhance hybridization and/or stability. Examples may include the use of nucleoside analogs, 5′ or 3′ overhangs on dsRNA or siRNA molecules, etc. See, e.g., U.S. patent application Ser. Nos. 11/750,553, 11/978,457, 11/746,864, 11/978,455, 11/978,398, 11/598,052, and 10/597,431, for a description of current techniques for synthesizing, modifying, and/or formulationg dsRNA or siRNA compositions, the disclosures of which are hereby incorporated by reference in their entirety.

Compositions of the present invention may further include DNA sequences which encode for each of the complementary dsRNA or siRNA strands. Such DNA compositions may be used to transiently or stably transfect host cells to express the dsRNA or siRNA molecules to treat or prevent viral infection in such cell through down-regulation of a target caspase gene. In addition, it is envisioned that gene therapy methods or viral vectors could be used to stably integrate such dsRNA or siRNA expressing constructs into the genome of a host cell to achieve the same.

According to another broad aspect of the present invention, methods are provided for the prevention, treatment, and/or management of viral infection. Such methods generally comprise methods of administering any of the compositions identified above, or a combination thereof, to patients or animals experiencing or at risk of viral infection.

According to another broad aspect of the present invention, screening methods are provided for identifying molecules or drug compounds from a library that are effective for the prevention, treatment, and/or management of viral infection or disease (i.e., to develop safe and effective inhibitors of viral propagation). Such molecules or drug compounds may be selected based on their ability to inhibit caspase activity. Such molecules or drug compounds may be further selected based on their ability to impede or block viral infection of host cells.

Compositions and methods of the present invention may be used to treat, prevent, and/or manage infection and/or disease caused by a variety of animal and human viruses acting either alone or in combination with other viruses in cases of simultaneous or subsequent infection. As described above, compositions of the present invention show promise against a broad spectrum of viruses since compositions of the present invention target host cell caspases that are commonly utilized by such viruses. FGI-103 compounds have been shown to be effective inhibitors of viruses encompassing multiple and varied virus families including, for example, respiratory syncytial virus (RSV), parainfluenza (PIV), pox viruses, Ebola, Marburg, Rift Valley fever, Lassa fever, PRRS, and others. Further examples of viruses inhibited by FGI-103 compounds are described in U.S. application Ser. Nos. 11/952,421 and 60/982,227, the disclosure of which is hereby incorporated by reference.

Results thusfar have suggested that FGI-103 compounds may be effective in inhibiting infection by all groups of viruses (defined according to the Baltimore classification). See, e.g., the description provided by U.S. App 60/982, 227, the disclosure of which is hereby incorporated by reference. Without being bound to any one theory, it is proposed that caspases may be involved during the early uncoating of the virus particle during infection to release viral contents into the host cell. Therefore, viruses that rely on caspase function to allow release of viral contents may have caspase consensus sequences (described above) displayed by proteins on their surface. Accordingly, compositions of the present invention may be effective in preventing, treating, and/or managing any virus displaying such consensus sequence on their surface. However, it is to be understood that such limitation is merely theoretical, and remains possible that either (i) caspases may “act on” viruses and cleave viral proteins not displaying any known consensus sequence on their surface, and/or (ii) caspases may inhibit viral infection indirectly. Therefore, compositions of the present invention are meant to include any caspase inhibitor that is also effective at inhibiting viral infection by a particular virus or set of viruses.

### EXAMPLES

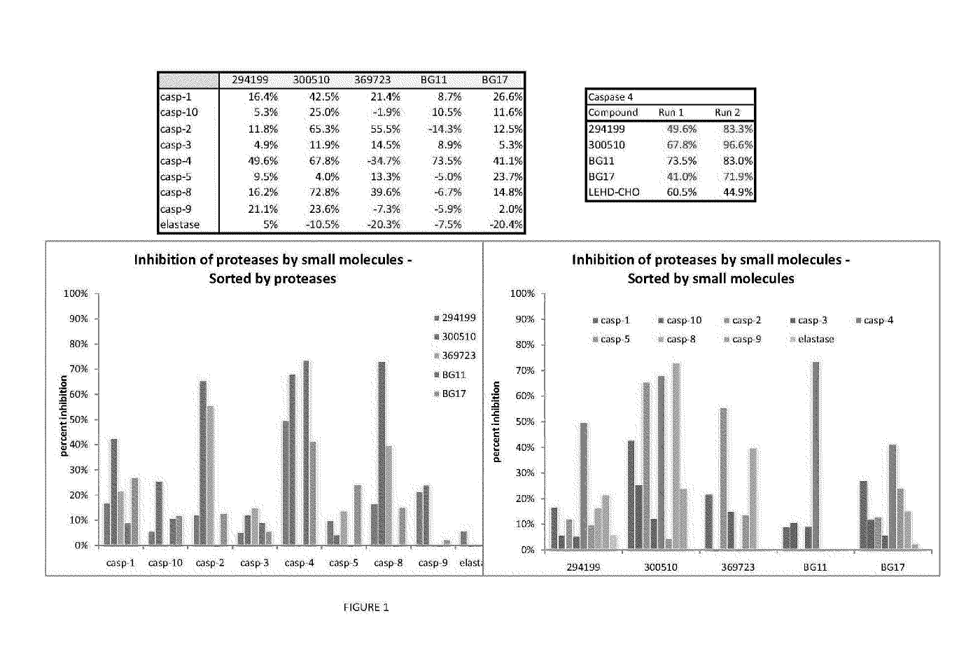

As representative examples of compounds having a common pharmacophore as FGI-103 compounds, NSC 294199, 300510, and 369723 were tested for their ability to inhibit each of the caspase enzymes The results are shown in Table 1 and FIG. 1. From these experiments, it is shown that the FGI-103 family of compounds effectively inhibit caspase enzymes, albeit to different extents. As shown in Table 2, the degree of inhibition by the FGI-103 compounds is roughly equivalent to a known caspase inhibitor, LEHD-CHO.

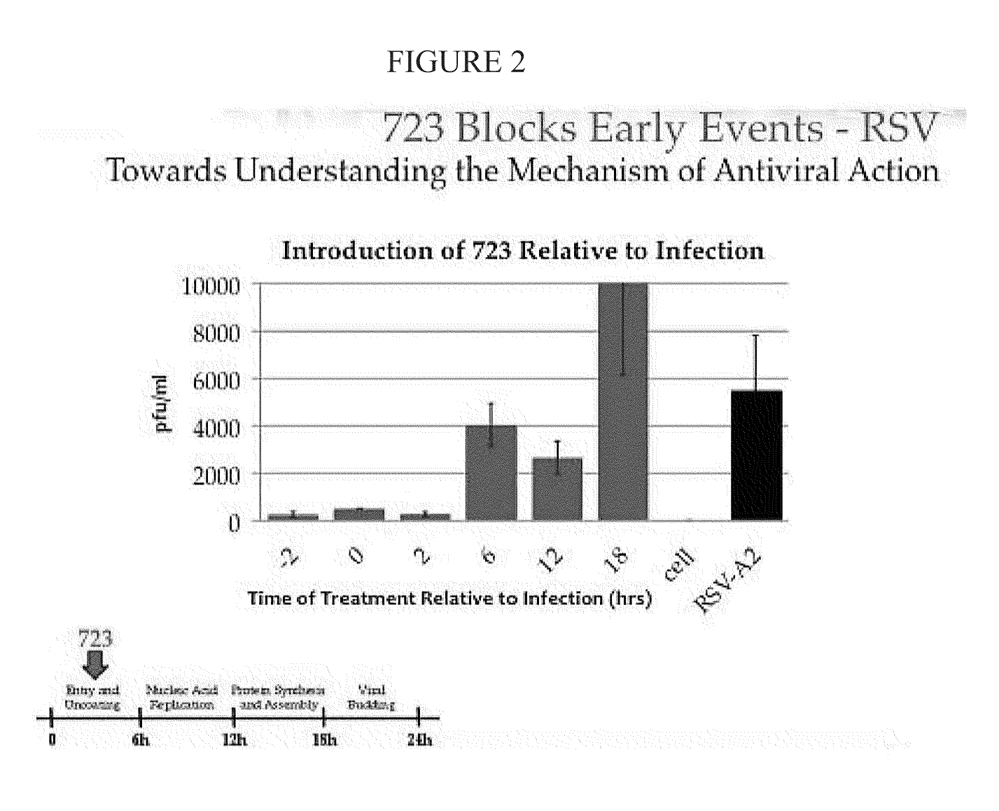

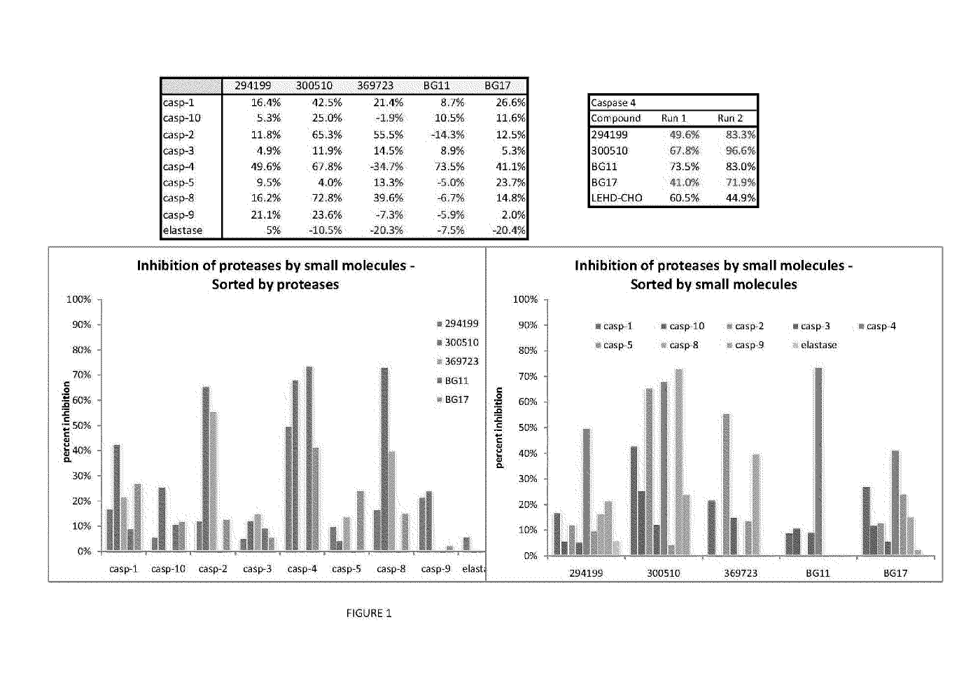

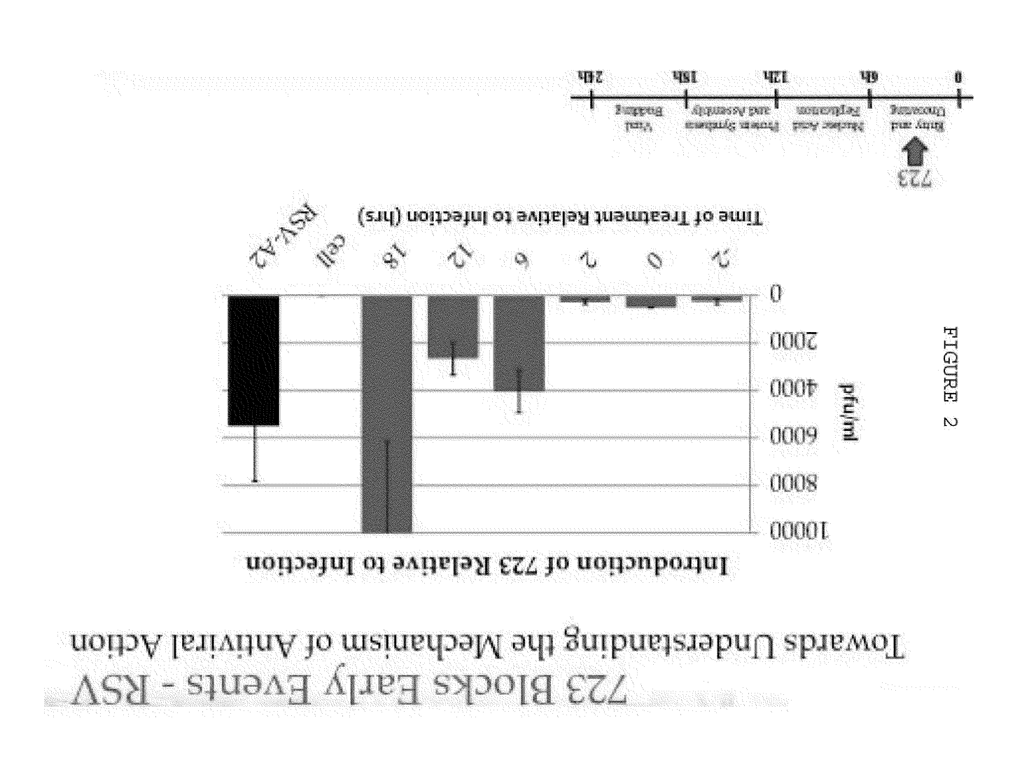

To determine the time in which treatment of cells with FGI-103 family compounds would be effective at inhibiting viral infection, NSC 369723 compound was tested for its ability to inhibit the production of virus particles (pfu/ml) depending on the time of treatment relative to the timing of infection of RSV. As shown by the experiment in FIG. 2, administration of the 723 compound either before or within the first few hours after infection effectively inhibited the production of virus particles. Furthermore, administration of the compound from about 6-12 hours moderately inhibited the production of viral particles, but by roughly 18 hours, the inhibitory effects were much reduced or lost.

As further shown by FIG. 2, the timing of effective treatment of viral infection was during the first few hours after viral infection corresponding to the timing for entry and uncoating of the virus particle. Without being bound to any one theory, it is thought that since viral attachment and uncoating occur within this timeframe and caspases are known to degrade proteins, the mechanistic basis by which FGI-103 compounds block viral infection could involve degradation of the viral coat (i.e., the surrounding envelope proteins of viruses) or other proteins to facilitate liberation of the viral genome. This mechanism is consistent with evidence that viral uncoating generally relates to conditions of low pH (e.g., the acidic environment of endosomes) and low pH appears to increase caspase activity. However, as stated above, such mechanism is merely theoretical. It remains possible that caspase inhibitors may interfere with viral infection through cleavage and/or activity involving independent or indirect mechanisms.

What is claimed is:

1. A composition, comprising: a caspase inhibitor, wherein said caspase inhibitor substantially inhibits viral infection caused by a virus in a host cell.

2. The composition of claim 1, wherein said caspase inhibitor is selected from the group consisting of NSC 294199, 300510, and 369723.

3. A pharmaceutical composition, comprising: an effective amount of the composition of claim 1.

4. The pharmaceutical composition of claim 3, further comprising a pharmaceutically suitable carrier or excipient.

5. A method of treatment, comprising: administering to a patient or animal the pharmaceutical composition of claim 3.

6. A method of screening, comprising: (i) selecting a compound that inhibits enzymatic activity of a caspase enzyme, and (ii) determining if said compound inhibits viral infection of a host cell.

7. A pharmaceutical composition, comprising: an effective amount of a compound identified by the screening method of claim 6.
